(19) United States
(12) Patent Application Publication
(10) Pub. No.: US 2017/0105953 A1
(43) Pub. Date: Apr. 20, 2017

(54) METHOD FOR SUPPRESSING DIABETES AND/OR HEPATIC LIPIDS USING TORMENTIC ACID

(71) Applicant: Chun-Ching Shih, Taichung City (TW)

(72) Inventors: Chun-Ching SHIH, Taichung City (TW); Jin-Bin WU, Taichung City (TW); Yueh-Hsiung KUO, Taipei City (TW); Cheng-Hsiu LIN, Taichung City (TW); Hui-Ya HO, Taichung City (TW)

(21) Appl. No.: 14/882,890

(22) Filed: Oct. 14, 2015

(51) Int. Cl. A61K 31/191

(52) U.S. Cl. CPC A61K 31/191

(57) ABSTRACT

Provided is a method for suppressing diabetes and/or hepatic lipids in a mammal to lower blood glucose levels and hepatic total lipids and triacylglycerol contents by increasing AMP-activated protein kinase (AMPK) phosphorylation in both skeletal muscle and liver tissue, and Akt phosphorylation and membraneprotein levels of glucose transporter 4 (GLUT4) in skeletal muscle. The method comprises administrating to the mammal an effective amount of tormentic acid or a pharmaceutically acceptable salt thereof, and a pharmaceutically acceptable carrier.

### BACKGROUND OF THE INVENTION

1. Field of the Invention

The present invention relates to a method for suppressing type 2 diabetes and/or hepatic lipids in a mammal, particularly suppressing diabetes and/or hepatic lipids by increasing the protein levels of glucose transporter 4 (GLUT4) in skeletal muscle, and expression levels of AMP-activated protein kinase (AMPK) phosphorylation in both skeletal muscle and liver tissue.

2. The Prior Arts

Type 2 diabetes represents >90% of all diabetes cases. Insulin resistance is found in the majority of type 2 diabetes caused by insensitivity to insulin in peripheral tissues. It is predicted that the prevalence of type 2 diabetes in the world's population will reach 6.1% by 2025.Therefore, finding a safer and less toxic substitute in the treatment of type 2 diabetes mellitus becomes important. Type 2 diabetes mainly reduces glucose uptake. Type 2 diabetes is accompanied by several complications causing a series of metabolic diseases including obesity and dyslipidemia. It is known that blood glucose and lipid constitute fluctuating homeostasis. Thus, finding a good resolution of glucose uptake and hepatic gluconeogenesis is an important issue in type 2 diabetes.

Insulin is secreted after a meal and, followed by glucose transporter 4 (GLUT4), is translocated to the plasma membrane, thus leading to glucose uptake into cells and contributing to reduced blood glucose. Insulin resistance and hyperglycemia are caused by problems in GLUT4 translocation and uptake. Thus, it is an important issue to increase protein contents and/or translocation of GLUT4 in the management of diabetes.

AMP-activated protein kinase (AMPK) regulates various metabolic pathways, and it is considered as an important target for the management of metabolic diseases including type 2 diabetes and dyslipidemia. Type 2 diabetes is found to be dysfunctional in glucose and lipid metabolism; therefore, AMPK modulators have been suggested to be promising therapies.

The plant Eriobotrya japonica Lindl. is an evergreen fruit tree and belongs to the Rosaceae family. The most used part of this plant is the dried leaf to treat diabetes mellitus. It is composed of many pentacyclic triterpenes, which demonstrate various pharmaceutical effects including hepatoprotection and antidiabetes. Callus tissue culture of loquat is reported to produce large amounts of triterpenes. Recently, it has shown that loquat leaf extract as well as its cell suspension culture (which contains five main bioactive constituents including tormentic acid (PTA) could improve insulin sensitivity and hepatic lipids; we think it is possible that the five constituents act synergistically on diabetes and lipid. Nevertheless, the effect of single and pure PTA of antidiabetes and antihyperlipidemia is still not fully understood.

### SUMMARY OF THE INVENTION

As a result, the present invention provides a method for suppressing diabetes in a mammal using only tormentic acid, having the chemical structure as Formula (I), contained in the loquat leaf extract. As having the effect of lowering the blood glucose, a method for treating or preventing diabetes and being an active ingredient of pharmaceutical composition can be achieved.

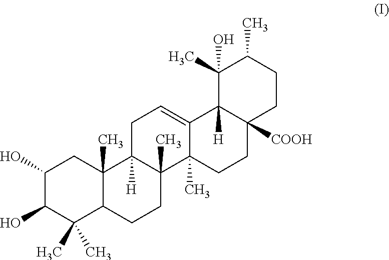

Another aspect of the present invention is to provide a method for suppressing hepatic lipids in a mammal using tormentic acid. Owing to the effects of decreasing hepatic total lipid and triacylglycerol contents, PTA can also be a component of a pharmaceutical composition for treating fatty liver.

Another aspect of the present invention is to provide a method for decreasing hepatic ballooning degeneration in a mammal using tormentic acid.

Another aspect of the present invention is to provide a method for suppressing diabetes and/or hepatic lipids in a mammal using tormentic acid by increasing skeletal muscular AMP-activated protein kinase (AMPK) phosphorylation.

Another aspect of the present invention is to provide a method for suppressing diabetes and/or hepatic lipids in a mammal using tormentic acid by increasing the expression levels of glucose transporter 4 (GLUT4).

Another aspect of the present invention is to provide a method for suppressing diabetes in a mammal using tormentic acid by increasing skeletal muscular Akt phosphorylation so as to increase insulin sensitivity.

As such, the present invention provides a method for_suppressing diabetes and/or hepatic lipids in a mammal, which comprises administrating to the mammal an effective amount of a compound of Formula (I) or a pharmaceutically acceptable salt thereof, and a pharmaceutically acceptable carrier.

In one embodiment, the present invention provides a pharmaceutical composition for suppressing or treating diabetes and/or hepatic lipids in a mammal, which comprises an effective amount of the compound of Formula (I) or a pharmaceutically acceptable salt thereof, and a pharmaceutically acceptable carrier.

In another embodiment, the present invention provides a use of the compound of Formula (I) or a pharmaceutically acceptable salt thereof in the manufacture of a medicament for suppressing diabetes and/or hyperlipidemia in a mammal.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1A shows the effects of tormentic acid on oral glucose tolerance test (OGTT) in normal mice;

FIG. 1B shows the effects of tormentic acid (PTA) onblood glucose levels at week 12;

FIG. 1C shows the effects of tormentic acid (PTA) on circulating triglyceride levels at week 12;

FIG. 2A shows the effects of tormentic acid (PTA) on epididymal WAT morphology in the low-fat (CON), high-fat (HF), HF +PTA1, HF +PTA2, or HF +Rosi groups;

FIG. 2B shows the effects of tormentic acid (PTA) on liver tissue morphology in the low-fat (CON), high-fat (HF), HF +PTA1, HF +PTA2, or HF +Rosi groups;

FIG. 3A is a electrophoresis picture of semiquantative RT-PCR analysis on PEPCK, G6 Pase, 11β-HSD1, DGAT2, PPARα, SREBP1c, FAS, and apo C-III mRNA levels in liver tissue of the mice receiving oral gavage extracts of tormentic acid (PTA) for 4 weeks;

FIG. 3B is a diagram showing the values quantitated and normalized by GAPDH of PEPCK, G6 Pase, 11β-HSD1, and DGAT2 from FIG. 3A;

FIG. 3C is a diagram showing the values quantitated and normalized by GAPDH of PARα, SREBP1c, FAS, and apo C-III from FIG. 3A;

FIG. 4A is a western blot analysis showing the protein contents of GLUT4 in skeletal muscle, and phospho-AMPK (Thr172) and total-AMPK (Thr172) in both liver tissue and skeletal muscle, and phospho-Akt (Ser473) and total-Akt (Ser473) in skeletal muscle of the mice receiving tormentic acid (PTA) by oral gavage for 4 weeks; and

FIG. 4B is a diagram showing the protein contents of GLUT4 in skeletal muscle, the expression levels of phospho-AMPK (Thr172) to total AMPK in both liver tissue and skeletal muscle, and quantified results from FIG. 4A for the phosphorylation status of Akt (p-Akt normalized to total Akt (pAkt/Akt)) in skeletal muscle of the mice receiving tormentic acid (PTA) by oral gavage for 4 weeks.

### DESCRIPTION OF THE PREFERRED EMBODIMENT

### Definition and General Terminology

As used herein, a “pharmaceutically acceptable salt” refers to organic or inorganic salts of a compound of the invention. Pharmaceutically acceptable salts are well known in the art. Some non-limiting examples of pharmaceutically acceptable, nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

The phrase “pharmaceutically acceptable” indicates that the compound, raw material, composition and/or dose must be compatible within a reasonable range of medical judgment and, when contacting with tissues of patients, is without overwhelming toxicity, irritation, transformation, or other problems and complications that are corresponsive to reasonable benefit/risk, while being effectively applicable for the predetermined purposes.

As used herein, the term “therapeutically effective amount” means the amount of a compound that, when administered to a mammal for treating a disease or a condition, is sufficient to effect such treatment for the disease or the condition. The “therapeutically-effective amount” will vary depending on the compound, the disease, and its severity and the age, weight, etc., of the mammal to be treated.

The following description and examples illustrate a preferred embodiment of the present invention in detail. Those of skill in the art will recognize that there are numerous variations and modifications of this invention encompassed by its scope. Accordingly, the description of a preferred embodiment should not be deemed to limit the scope of the present invention.

Tormentic acid (PTA) obtained from suspension cells of E. japonica has the effects of activating AMP-activated protein kinase (AMPK) phosphorylation and promoting the protein levels of glucose transporter 4 (GLUT4) gene, so that the blood glucose can be reduced and relieve the symptoms of diabetes. Furthermore, as mRNA levels of sterol regulatory element binding protein-1c (SREBP-1c), fatty acid synthase (FAS), and apolipoprotein C-III (apo C-III) are down-regulated by PTA in liver, triglycerides are finally reduced due to the suppression of de novo lipogenesis; whereas peroxisome proliferator activated receptor α(PPARα) is up-regulated and facilitates fatty acid oxidation. Accordingly, triglycerides in blood and liver can significantly be decreased.

Hereinafter, the present invention will be further illustrated with reference to the following examples. However, these examples are only provided for illustration purposes, but not to limit the scope of the present invention.

### EXAMPLE 1

### Preparation of Tormentic Acid

Tormentic Acid (PTA) was obtained from Jen Li Biotech Co. Callus induction, suspension cultures, and extraction and isolation of tormentic acid from suspension cells of E. japonica were performed as previously described. Briefly, sterilized seeds after callus induction were cultured in a bioreactor, and the cell suspension (ca. 844.5 g) was dried and extracted with ethanol and then concentrated to afford the white powder fraction (ca. 6.1 g). The white powder (0.5 g) was chromatographed on a reverse silica gel column (LiChroprep RP-18, E. Merck, 40-63 μm) and then further purified by preparative high-performance liquid chromatography (PHPLC) to yield tormentic acid.

The cell suspension described above can be extracted by, but not limited to ethanol, methanol or other alcohols used in the art.

The purified tormentic acid was analyzed by mass spectrometry and NMR, and recognized as the following compound of Formula (I). Tormentic acid (230.5 mg): ¹H NMR (pyridine-d5) δ 1.00 (H-25), 1.07 (H-24), 1.10 (H-26), 1.11 (H-30), 1.26 (H-23), 1.42 (H-29), 1.71 (H-27), 3.04 (H-18), 3.36 (H-3α), 4.09 (H-3β), 5.58 (H-12). ¹H NMR (400 MHz) spectra were measured by using a Bruker AMX-400 spectrometer as previously described.

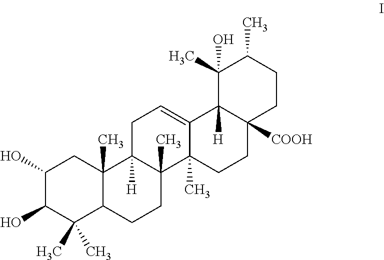

### EXAMPLE 2

### Animals and Experimental Design

### (1) Animals

Owing to the case that the mouse C57BL/6 model fed with a high-fat (HF) diet could induce insulin resistance, obesity, hyperlipidemia, hyperinsulinemia, hypertriglyceridemia, and excess circulating free fatty acid, the animal study is conducted by using HF diet-induced diabetic and hyperlipidemic states.

Moreover, AMPK activity is dependent on the phosphorylation of Thr 172 of α subunits. Thus the present invention also examined the effect of PTA on the expression of genes or proteins involved in antidiabetes and lipogenesis, including GLUT4, p-Akt, p-AMPK, phosphoenol pyruvate carboxykinase (PEPCK), glucose-6-phosphatase (G6 Pase), sterol regulatory element binding protein-1c (SREBP-1c), peroxisome proliferator-activated receptor α (PPARα), and apolipoprotein C-III (apo-CIII).

### (2) Oral Glucose Tolerance Test

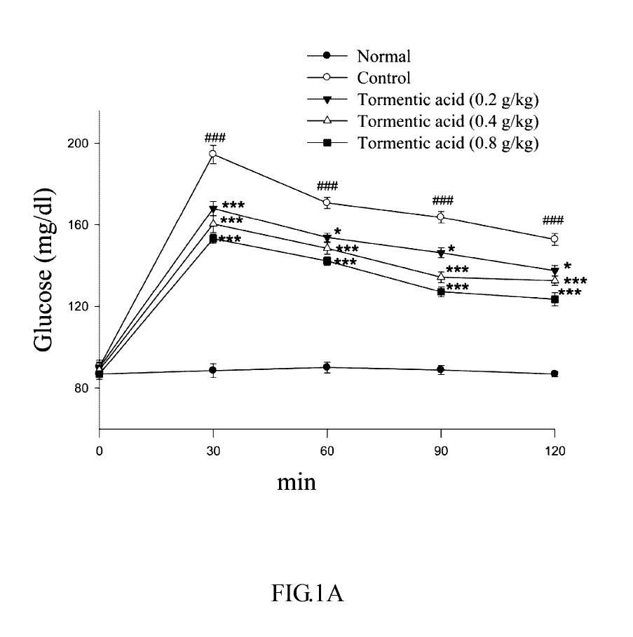

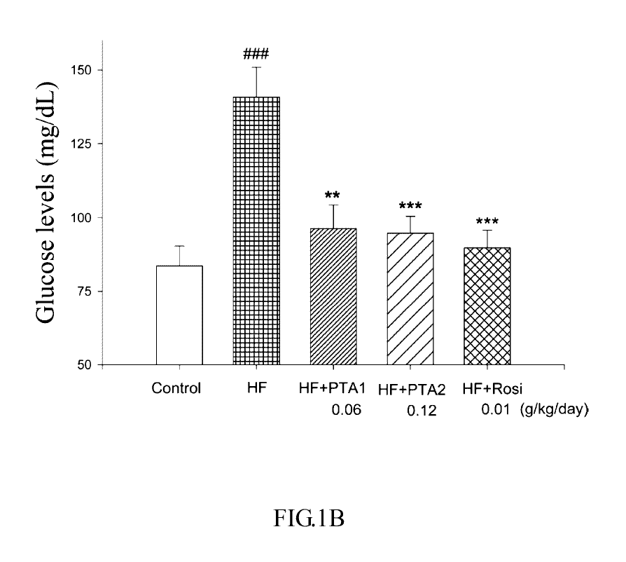

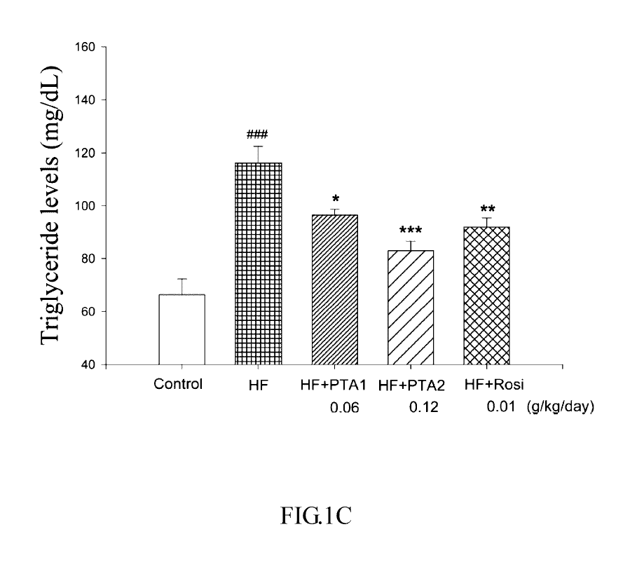

For part 1, an oral glucose tolerance test (OGTT) was performed on 12 h fasted ICR mice (n=5) that were allowed access to 0.2, 0.4, and 0.8 g/kg PTA or an equivalent amount of vehicle (water), which were given orally 30 min before an oral glucose load (1 g/kg body wt). The control group was given glucose, whereas the normal group was not. Blood samples were collected from the retro-orbital sinus of fasted mice at the time of the glucose administration (0) and every 30 min until 120 min after glucose administration. The blood glucose level was monitored, and the result is shown in FIG. 1A.

As shown in FIG. 1A, the levels of blood glucose by administration of 0.2, 0.4, and 0.8 g/kg tormentic acid are decreased from 30 to 120 min following a glucose loading.

For part 2, C57BL/6J mice (4 weeks old) were obtained from the National Laboratory Animal Breeding and Research Center. The mice were divided randomly into two groups after 7 days acclimation. The control (CON) group (n=9) was fed a low-fat diet (diet 12450B, Research Diets, Inc., New Brunswick, N.J., USA), whereas the experimental group was fed a 45% high-fat diet (diet 12451, Research Diets, Inc.) for 12 weeks. The compositions of the experimental diets are given in previous studies. After 8 weeks, the high-fat treated mice were randomly subdivided into four groups (n=9) including PTA1 (0.06 g/kg/day), PTA2 (0.12 g/kg/day) or rosiglitazone (Rosi; 1% methylcellulose, 10 mg/kg body weight) (GlaxoSmithKline) or vehicle and treated by oral gavage one time per day from the 9th to 12th weeks, while the mice were still on the high-fat diet, whereas the CON and high-fat control (HF) mice were treated with vehicle only. At the end, food was withheld from the animals (from 10 p.m. to 10 a.m.). The next day, the mice were sacrificed for blood and tissue collection and analysis. Livers, skeletal muscles, and white adipose tissues (WATs) (including epididymal, mesenteric, and retroperitoneal WAT) were weighed and excised, followed by immediate freezing, and kept at −80° C. for target gene analysis. Heparin (30 units/mL) (Sigma) was added to blood samples. Plasma samples were collected within 30 min by centrifugation at 1600 g for 15 min at 4° C. Plasma was obtained for insulin and leptin assay.

### (3) Analysis of Blood Parameters.

Blood samples (0.8 mL) were collected from the retro-orbital sinus of fasted mice, and the glucose level was analyzed by the glucose oxidase method (model 1500; Sidekick Glucose Analyzer; YSI Inc., Yellow Springs, Ohio, USA). Plasma triglycerides (TG), total cholesterol (TC), and free fatty acids (FFA) were measured using commercial assay kits according to the manufacturer's directions (Triglycerides-E test, Cholesterol-E test, and FFA-C test, Wako Pure Chemical, Osaka, Japan). The levels of insulin and leptin in blood were analyzed by ELISA using a commercial assay kit according to the manufacturer's directions (mouse insulin ELISA kit, Sibayagi, Gunma, Japan; and mouse leptin ELISA kit, Morinaga, Yokohama, Japan).

For part 2, as shown in FIG. 1B, mice fed with a high-fat (HF) diet for 12 weeks have a mean blood glucose of 140.8 mg/dL, which is significantly more than the value of 83.6 mg/dL (P<0.001) of mice fed with a low-fat diet. In comparison, the blood glucose values of mice in experimental groups treated with PTA1, PTA2 and Rosi are reduced to 96.3 mg/dL, 94.8 mg/dL and 89.7 mg/dL (P<0.001; P<0.001; P<0.001). That is to say, HF increases blood glucose, whereas administration of PTA1, PTA2, and Rosi lower blood glucose levels.

Blood parameters, leptin, insulin and liver lipids measured are shown in Table 1 and FIG. 1C. All values are means ±SE (n=9). #P<0.05; ##P<0.01; and ###P<0.001 compared with the control (CON) group. *P<0.05; **P<0.01; and ***P<0.001 compared with the high-fat+vehicle (distilled water) (HF) group. Tormentic acid (PTA1:0.06 and PTA2:0.12 g/kg body wt); Rosi, rosiglitazone (0.01 g/kg body wt); BAT, brown adipose tissue; RWAT, retroperioneal white adipose tissue; MWAT, mesenteric white adipose tissue; visceral fat, sum of epididymal and retroperioneal WAT; FFA, plasma free fatty acid; TC, total cholesterol; TG, triglyceride.

TABLE 1

| | | | | | |
|---|---|---|---|---|---|
| | | | HF + PTA1 | HF + PTA2 | HF + Rosi |
| Parameter | CON | HF | 0.06 g/kg/day | 0.12 g/kg/day | 0.01 g/kg/day |

Liver lipids

| | | | | | |
|---|---|---|---|---|---|
| Total lipid | 55.2 ± 5.1 | 98.7 ± 6.4### | 80.1 ± 4.6* | 68.2 ± 5.9** | 74.7 ± 5.7** |
| (mg/g) | | | | | |
| Triacylglycerol | 31.5 ± 4.2 | 68.5 ± 7.1### | 51.7 ± 5.1* | 35.2 ± 4.9*** | 43.8 ± 4.7*** |
| (μmol/g) | | | | | |

Blood profiles

| | | | | | |
|---|---|---|---|---|---|
| FFA (mequiv/L) | 1.63 ± 0.14 | 2.75 ± 0.42# | 2.15 ± 0.35* | 1.88 ± 0.29* | 1.85 ± 0.28* |
| TC (mg/dL) | 87.7 ± 5.7 | 188.5 ± 10.2### | 167.0 ± 6.1 | 153.7 ± 6.9* | 140.7 ± 14.1* |
| Leptin (μg/mL) | 1.523 ± 0.072 | 3.124 ± 0.065### | 2.345 ± 0.092*** | 1.780 ± 0.094*** | 2.151 ± 0.099*** |
| Insulin (μU/mL) | 31.3 ± 5.9 | 162.6 ± 16.5### | 101.7 ± 15.2* | 73.9 ± 11.8** | 56.4 ± 13.8** |

As seen from Table 1 and FIG. 1C, high-fat diets (HF) cause increases in circulating TG, FFA, leptin, and insulin levels compared therewith of low-fat diets, namely the PTA1-, PTA2-, and Rosi-treated mice display decreased TG, FFA, leptin, and insulin. In addition, the PTA2- and Rosi-treated mice show decreased TC levels. Moreover, the HF diet increases the total lipids of liver (up to 98.7 mg/g) and concentrations of triacylglycerol (up to 68.5 μmol/g), whereas mice administrated PTA1, PTA2, and Rosi show significant decreases in these phenomena; particularly, treated with 0.12 g/kg PTA (PTA2) can effectively reduce the concentration of triacylglycerol to 35.2 μmol/g (P<0.001).

### (4) Analysis of Histopathology.

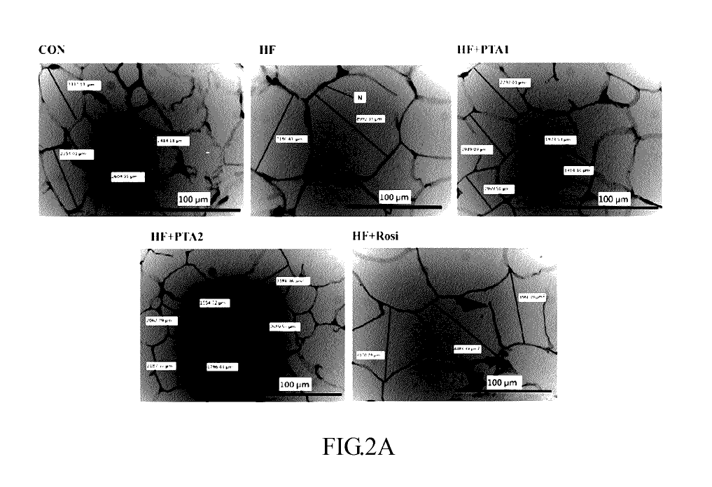

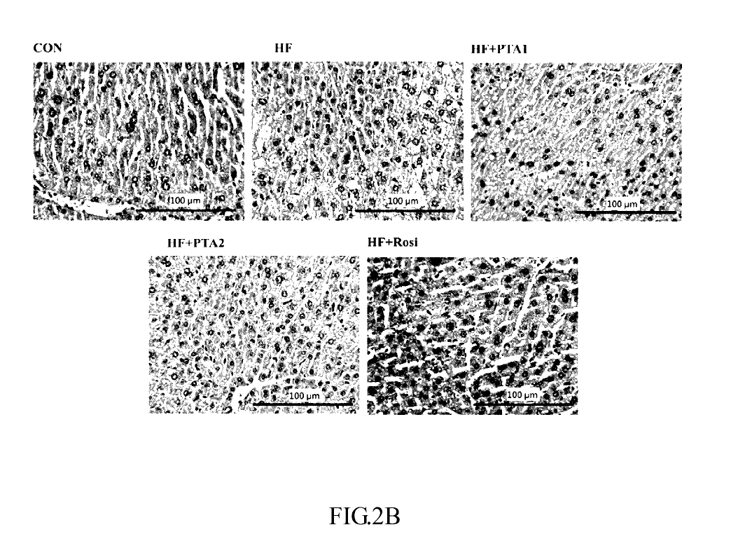

Small pieces of epididymal WAT and liver tissue were fixed with formalin (200 g/kg) neutral buffered solution and embedded in paraffin. Sections (8 μm) were cut and stained with hematoxylin and eosin. For microscopic examination, a microscope (Leica, DM2500) was used, and the images were taken using a Leica Digital camera (DFC-425-C). The results are shown in FIGS. 2A and 2B.

As shown in FIG. 2A, HF induces adipocyte hypertrophy (the average areas of adipocytes in the HF group and CON group are 6515.9±495.1 and 2584.6±205.8 μm², respectively), whereas mice administered PTA1 (2380.9±108.6 μm2) and PTA2 (2243.2±100.9 μm²) show significantly lower hypertrophy. The average area of the Rosi treated mice is 4574.4±162.7 μm². According to a previous study, designation of histological hepatocellular ballooning findings included grade 0, none; grade 1, a few cells; grade 2, many cells. The ballooning phenomenon in liver as seen from FIG. 2B is visible on the HF diet (mean score=1.7±0.2); by contrast the ballooning phenomenon is lower in the PTA1-treated (1.0±0.2), PTA2-treated (0.7±0.2), and Rosi-treated (0.9±0.2) mice.

### (5) Analysis of Hepatic Lipids.

Hepatic lipids were extracted using a previously described protocol. For the hepatic lipid extraction, 0.375 g liver samples were homogenized with 1 mL of distilled water for 5 min. Finally, the dried pellet was resuspended in 0.5 mL of ethanol and analyzed using a triglycerides kit as used for serum lipids. The result is shown in FIG. 2C.

(6) Isolation of RNA and Relative Quantization of mRNA Indicating Gene Expression

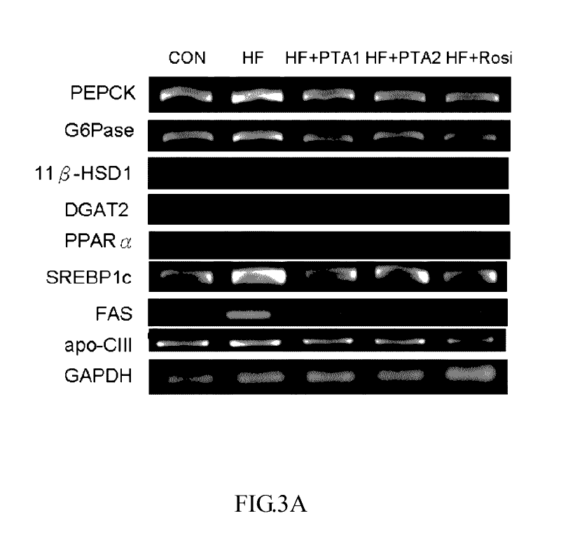

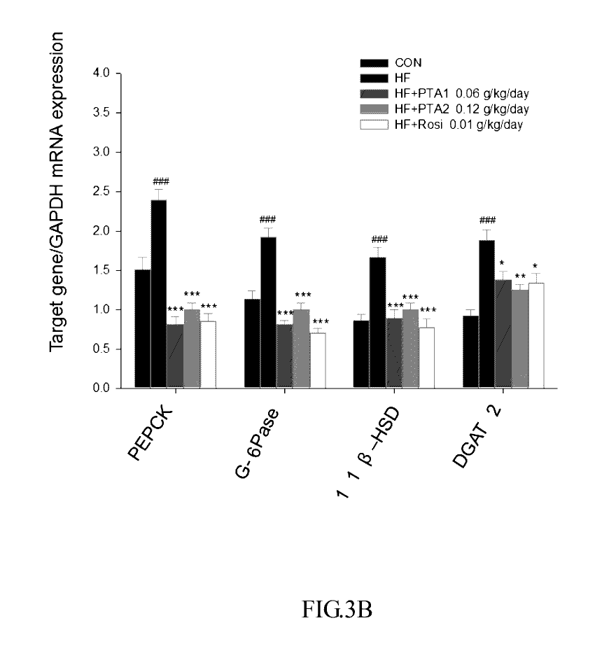

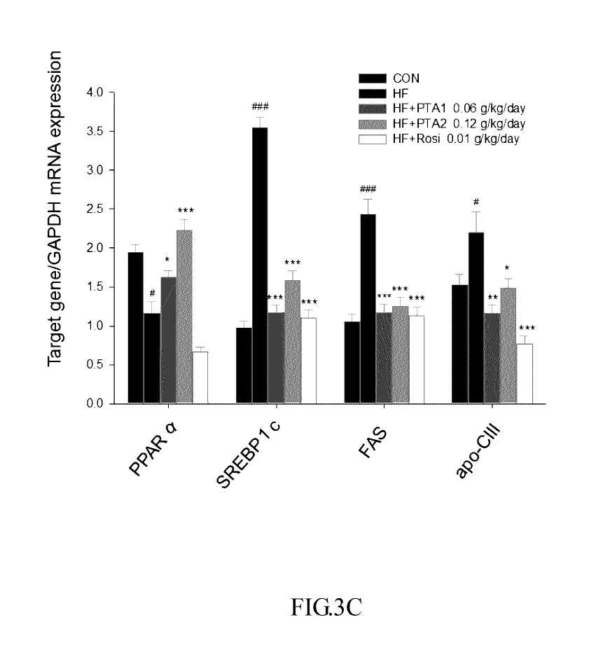

Total RNA from the liver tissue was isolated with a Trizol reagent (Molecular Research Center, Inc., Cincinnati, Ohio, USA) according to the manufacturer's directions. The integrity of the extracted total RNA was examined by 2% agarose gel electrophoresis, and the RNA concentration was determined by ultraviolet (UV) light absorbency at 260 and 280 nm (spectrophotometer U-2800A, Hitachi). Total RNA (1 μg) was reverse transcribed to cDNA with 5 μL of Moloneymurine leukemia virus reverse transcriptase (Epicenter, Madison, Wis., USA) as a previously described protocol. The polymerase chain reaction (PCR) was performed in a final 25 μL containing 1 U of Blend Taq-Plus (TOYOBO, Japan), 1 μL of the RT first-strand cDNA product, 10 μM of each forward (F) and reverse (R) primer, 75 mM Tris-HCl (pH 8.3) containing 1 mg/L Tween 20, 2.5 mM dNTP, and 2 mM MgCl₂. The primers are shown in Table 2. The products were run on 2% agarose gels and stained with ethidium bromide. The relative density of the band was evaluated using AlphaDigiDoc 1201 software (Alpha Innotech Co., San Leandro, Calif., USA). All of the measured PCR products were normalized to the amount of cDNA of GAPDH in each sample. The results are shown in FIGS. 3A, 3B and 3C (All values are means ±SE (n=9). #P<0.05, ##P<0.01, and ###P<0.001 compared with the control (CON) group; *P<0.05, **P<0.01, and ***P<0.001 compared with the high-fat plus vehicle (distilled water) (HF) group by ANOVA. Tormentic acid (PTA1:0.06 and PTA2:0.12 g/kg body wt); Rosi, rosiglitazone (0.01 g/kg body wt); WAT, white adipose tissue; epididymal WAT+retroperitoneal WAT, visceral fat. Pathological Diagnosis).

TABLE2

| | | | PCR | Annealing |
|---|---|---|---|---|
| | | Forward | Product | Temp |
| Gene | Accession No. | And Reverse Primers | (bp) | (° C.) |
| PEPCK | NM_011044.2 | F: CTACAACTTCGGCAAATACC | 330 | 52 |
| | | (SEQ ID NO: 1) | | |
| | | R: TCCAGATACCTGTCGATCTC | | |
| | | (SEQ ID NO: 2) | | |
| G6 Pase | NM_008061.3 | F: GAACAACTAAAGCCTCTGAAAC | 350 | 50 |
| | | (SEQ ID NO: 3) | | |
| | | R: TTGCTCGATACATAAAACACTC | | |
| | | (SEQ ID NO: 4) | | |
| 11β-HSD1 | NM_008288.2 | F: AAGCAGAGCAATGGCAGCAT | 300 | 50 |
| | | (SEQ ID NO: 5) | | |
| | | R: GAGCAATCATAGGCTGGGTCA | | |
| | | (SEQ ID NO: 6) | | |
| DGAT2 | NM_026384.3 | F: AGTGGCAATGCTATCATCATCGT | 149 | 50 |
| | | (SEQ ID NO: 7) | | |
| | | R: AAGGAATAAGTGGGAACCAGATCA | | |
| | | (SEQ ID NO: 8) | | |
| PPARα | NM_011144 | F: ACCTCTGTTCATGTCAGACC | 352 | 55 |
| | | (SEQ ID NO: 9) | | |
| | | R: ATAACCACAGACCAACCAAG | | |
| | | (SEQ ID NO: 10) | | |
| SREBP1c | NM_011480 | F: GGCTGTTGTCTACCATAAGC | 219 | 50 |
| | | (SEQ ID NO: 11) | | |
| | | R: AGGAAGAAACGTGTCAAGAA | | |
| | | (SEQ ID NO: 12) | | |
| FAS | NM_007988 | F: TGGAAAGATAACTGGGTGAC | 240 | 50 |
| | | (SEQ ID NO: 13) | | |
| | | R: TGCTGTCGTCTGTAGTCTTG | | |
| | | (SEQ ID NO: 14) | | |
| apo | NM_023114.3 | F: CAGTTTTATCCCTAGAAGCA | 349 | 47 |
| C-III | | (SEQ ID NO: 15) | | |
| | | R: TCTCACGACTCAATAGCTG | | |
| | | (SEQ ID NO: 16) | | |
| GAPDH | NM_031144 | F: TGTGTCCGTCGTGGATCTGA | 99 | 55 |
| | | (SEQ ID NO: 17) | | |
| | | R: CCTGCTTCACCACCTTCTTG | | |
| | | (SEQ ID NO: 18) | | |

### Hepatic Target Gene Expressions

As shown in FIGS. 3A, 3B and 3C, the mRNA levels of PEPCK, G6 Pase, 11β-hydroxysteroid dehydrogenase 1 (11β-HDS1), diacyl glycerol acyltransferase 2 (DGAT2), PPARα, SREBP1c, fatty acid synthase (FAS), and apo C-III are increased in the HF group. Following treatment, the PTA1-, PTA2-, and Rosi-treated groups significantly decrease the mRNA level of PEPCK, G6 Pase, DGAT2, 11β-HSD1, SREBP1c, FAS, and apo C-III. The PTA1- and PTA2-treated groups show increased mRNA levels of PPARα.

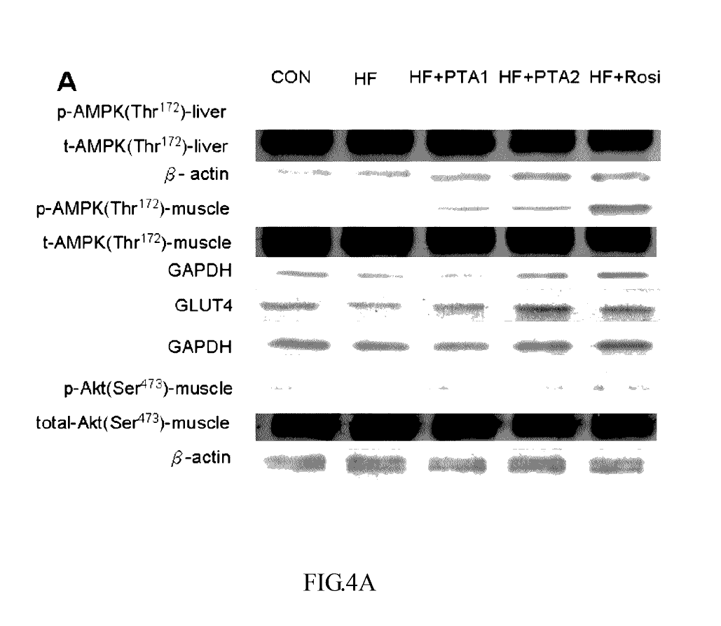

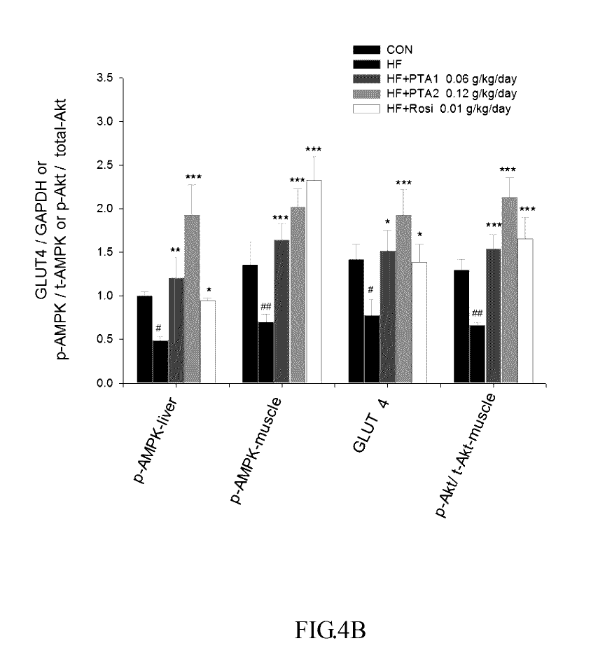

As shown in FIGS. 4A and 4B, the expression levels of phospho-AMPK/total-AMPK are decreased in the HF group, whereas the PTA1-, PTA2-, and Rosi-treated groups significantly increase the expression levels of phospho-AMPK/total-AMPK in both liver tissue and skeletal muscle. In HF-induced mice the expression levels of GLUT4 and phospho-Akt/total-Akt are decreased, whereas in PTA1-, PTA2-, and Rosi-treated mice the membrane protein levels of GLUT4 and expression levels of phospho-Akt/total-Akt in skeletal muscle are significantly increased. Therefore, the increased membrane protein level of GLUT4 promotes skeletal muscular glucose uptake, which contributed to reduce blood glucose levels.

The present invention shows that PTA2 causes marked increases of GLUT4 protein, and PTA1 displays GLUT4 levels similar to those of Rosi, which is an antidiabetic agent and directly targets insulin resistance and increases peripheral glucose uptake (GLUT4), leading to improved glycemic control. These findings are the first to reveal that PTA caused increased GLUT4 proteins as well as a direct relationship exhibiting antidiabetic activity. Thus, PTA displayed a very marked enhancement of GLUT4 accompanied by ameliorated insulin resistance.

There are two pathways in regulated GLUT4 translocation including insulin signaling (those involving phosphatidylinositol kinase (PI3K)/Akt) and the AMPK pathway. To monitor the mechanism of enhanced GLUT4 by PTA, the effects on the phosphorylation of Akt in the skeletal muscle are evaluated. The results demonstrated that PTA has a significantly increased effect on skeletal muscular phosphorylation of Akt, suggesting that PTA increased muscular GLUT4 contents are likely to be partly mediated by Akt phosphorylation; moreover, PTA enhanced Akt phosphorylation and increased insulin sensitivity.

The liver is the organ responsible for the majority of hepatic gluconeogenesis. A number of hormones regulate a set of genes (including PEPCK and G6 Pase) in the liver that modulates the rate of glucose synthesis. PEPCK is a rate controlling step of gluconeogenesis in animals, and G6 Pase plays a vital role in glucose homeostasis. Overexpression of the PEPCK enzyme in mice results in symptoms of type 2 diabetes. The hepatic G6 Pase activities of diabetic animals are increased. PTA treatment reduces the expressions of PEPCK and G6 Pase. Therefore, the antidiabetic effect of PTA is partly due to down-regulation of PEPCK and G6 Pase.

PEPCK is controlled by hormonal mechanisms including 11β-HSD1. 11β-HSD1 knockout mice fed a HF diet are protected from developing insulin resistance. Therefore, compounds that down-regulate 11β-HSD1 might contribute to antidiabetic activities. Besides PEPCK and G6 Pase, PTA decreases hepatic 11β-HSD1 expressions also lead to enhanced insulin sensitivity.

HF-induced reduced phospho-AMPK in the liver, with increased in PTA and Rosi-treated groups, indicates improved hyperglycemia by AMPK activation. Besides increasing muscular glucose uptake (GLUT4), PTA is likely to reduce hepatic glucose production by down-regulations of PEPCK and G6 Pase via AMPK.

To monitor the mechanism of PTA on ameliorating liver lipids, it is found that PTA increases PPARα mRNA levels. PPARα ligands (such as fibrates) have been shown to reduce the mRNA levels of apo C-III gene, thus resulting in lowering fat values in the blood and liver and exhibiting a hypotriglyceridemic effect. Furthermore, DGAT2 catalyzes the final step in the synthesis of triglycerides. It is found that PTA decreased circulating TG levels, and this may be partly associated with decreased DGAT2 mRNA levels.

Moreover, PTA suppresses the mRNA levels of FAS, which is a key enzyme in fatty acid synthesis. The glucose-induced SREBP1c and FAS mRNA levels are also down-regulated by AMPK. The AMPK activator metformin has been shown to down-regulate FAS expression via AMPK activation. Therefore, it is possible that PTA down-regulated these genes through AMPK activation. The evidence for morphological analysis comes from the finding that treatment with PTA1 and PTA2 decrease the hypertrophy of adipocytes. The liver is a major organ metabolizing fat. The level of circulating TG fluctuates; it is possible that PTA caused fat to move from adipose to liver tissue by increasing hepatic lipid catabolism, thus resulting in decreased size of adipocytes and almost the complete absence of liver lipid droplets.

In conclusion, the present invention shows that PTA effectively lowers hyperglycemia and hypertriglyceridemia in HF-fed mice. PTA improves glycemic control primarily via increased skeletal muscular GLUT4 proteins to elevate glucose uptake but suppresses hepatic glucose production (down-regulations of PEPCK and G6 Pase). PTA also enhances AMPK phosphorylation both in skeletal muscle and in the liver. In addition, PTA enhances skeletal muscular Akt phosphorylation and increased insulin sensitivity. Further, PTA increases hepatic fatty acid oxidation (PPARα), but suppresses lipogenic enzyme expression (including SREBP1c and FAS), thus contributing to lowering triglyceride levels. Consequently, tormentic acid is effective on type 2 diabetes and ameliorating hepatic lipids in HF-fed mammals including mice.

1. A method for suppressing type 2 diabetes and/or hepatic lipids in a mammal, comprising administrating to the mammal an effective amount of a compound of Formula (I) or a pharmaceutically acceptable salt thereof, and a pharmaceutically acceptable carrier:

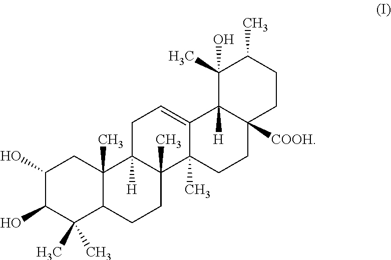

2. The method according to claim 1, wherein the compound suppresses type 2 diabetes and/or hepatic lipids by decreasing blood glucose levels and regulating blood insulin levels.

3. The method according to claim 1, wherein the compound suppresses type 2 diabetes and/or hepatic lipids by increasing AMP-activated protein kinase (AMPK) phosphorylation in both skeletal muscle and liver tissue.

4. The method according to claim 1, wherein the compound suppresses type 2 diabetes and/or hepatic lipids by increasing the expression levels of glucose transporter 4 (GLUT4).

5. The method according to claim 1, wherein the compound suppresses type 2 diabetes and/or hepatic lipids by increasing skeletal muscular Akt phosphorylation, and the increased skeletal muscular Akt phosphorylation increases insulin sensitivity.

6. The method according to claim 1, wherein the compound suppresses type 2 diabetes and/or hepatic lipids partly by decreasing the mRNA levels of phosphenolpyruvatecarboxykinase (PEPCK) and glucose-6-phosphatase (G6 Pase), and reducing hepatic glucose production.

7. The method according to claim 1, wherein the compound protects the mammal from high-fat diet-induced fatty liver by decreasing hepatic total lipid and triacylglycerol, and histologically ballooning degeneration of hepatocytes.

8. The method according to claim 1, wherein the compound suppresses type 2 diabetes and/or hepatic lipid partly by increasing the mRNA level of peroxisome proliferator activated receptor α (PPARα) and decreasing that of fatty acid synthase (FAS).

9. The method according to claim 1, wherein the compound enhances hepatic lipid catabolism and reduces hepatic total lipid, and which is followed by a reduction of circulating triglyceride, and a decrease of the area of adipocyte.

10. A pharmaceutical composition for suppressing type 2 diabetes and/or hepatic lipids in a mammal comprising the compound of claim 1 or a pharmaceutically acceptable salt thereof, and a pharmaceutically acceptable carrier.


SEQUENCE LISTING (18 sequences)

SEQ ID NO: 1 - DNA, 20 bp, Artificial Sequence
  PCR Primer
ctacaacttcggcaaatacc

SEQ ID NO: 2 - DNA, 20 bp, Artificial Sequence
  PCR Primer
tccagatacctgtcgatctc

SEQ ID NO: 3 - DNA, 22 bp, Artificial Sequence
  PCR Primer
gaacaactaaagcctctgaaac

SEQ ID NO: 4 - DNA, 22 bp, Artificial Sequence
  PCR Primer
ttgctcgatacataaaacactc

SEQ ID NO: 5 - DNA, 20 bp, Artificial Sequence
  PCR Primer
aagcagagcaatggcagcat

SEQ ID NO: 6 - DNA, 21 bp, Artificial Sequence
  PCR Primer
gagcaatcataggctgggtca

SEQ ID NO: 7 - DNA, 23 bp, Artificial Sequence
  PCR Primer
agtggcaatgctatcatcatcgt

SEQ ID NO: 8 - DNA, 24 bp, Artificial Sequence
  PCR Primer
aaggaataagtgggaaccagatca

SEQ ID NO: 9 - DNA, 20 bp, Artificial Sequence
  PCR Primer
acctctgttcatgtcagacc

SEQ ID NO: 10 - DNA, 20 bp, Artificial Sequence
  PCR Primer
ataaccacagaccaaccaag

SEQ ID NO: 11 - DNA, 20 bp, Artificial Sequence
  PCR Primer
ggctgttgtctaccataagc

SEQ ID NO: 12 - DNA, 20 bp, Artificial Sequence
  PCR Primer
aggaagaaacgtgtcaagaa

SEQ ID NO: 13 - DNA, 20 bp, Artificial Sequence
  PCR Primer
tggaaagataactgggtgac

SEQ ID NO: 14 - DNA, 20 bp, Artificial Sequence
  PCR Primer
tgctgtcgtctgtagtcttg

SEQ ID NO: 15 - DNA, 20 bp, Artificial Sequence
  PCR Primer
cagttttatccctagaagca

SEQ ID NO: 16 - DNA, 19 bp, Artificial Sequence
  PCR Primer
tctcacgactcaatagctg

SEQ ID NO: 17 - DNA, 20 bp, Artificial Sequence
  PCR Primer
tgtgtccgtcgtggatctga

SEQ ID NO: 18 - DNA, 20 bp, Artificial Sequence
  PCR Primer
cctgcttcaccaccttcttg